(19) United States
(12) Patent Application Publication
(10) Pub. No.: US 2024/0318203 A1
(43) Pub. Date: Sep. 26, 2024

(54) APPARATUSES AND METHODS FOR DIGITAL MICROFLUIDICS AND MICRO-ELECTROPORATION

(71) Applicant: mirOculus Inc., San Francisco, CA (US)

(72) Inventors: Mais J. JEBRAIL, Toronto (CA); Ryan MONTES, San Francisco, CA (US); An-Angela VAN, San Francisco, CA (US); Ik Pyo HONG, Toronto (CA); Eduardo CERVANTES, San Francisco, CA (US); Simin LIU, San Francisco, CA (US); Louis DALTCHEV, San Francisco, CA (US); Foteini CHRISTODOULOU, San Francisco, CA (US)

(21) Appl. No.: 18/568,534

(22) PCT Filed: Jun. 8, 2022
(86) PCT No.: PCT/US2022/032743

§ 371 Date: Dec. 8, 2023

Related U.S. Application Data
(60) Provisional application No. 63/208,479, filed on Jun. 8, 2021.

(51) Int. Cl. C12N 15/87; C12M 1/00; C12M 1/12; C12M 1/42; C12M 3/00; C12M 3/06

(52) U.S. Cl. CPC C12N 15/87; C12M 23/16; C12M 23/26; C12M 23/42; C12M 25/02; C12M 35/02

(57) ABSTRACT

Microfluidic apparatuses and methods of making and using them. In particular, described herein are microfluidic cartridges, such as digital microfluidic cartridges, including micro-electroporation electrodes and systems for using them in which droplets may be moved by electrowetting and electroporated in the same regions.

### CLAIM OF PRIORITY

This patent application claims priority to U.S. Provisional Patent Application No. 63/208,479, titled “APPARATUSES AND METHODS FOR DIGITAL MICROFLUIDICS AND MICRO-ELECTROPORATION” and filed on Jun. 8, 2021, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Gene editing is becoming more promising in the development of new genomic biomarkers to predict disease risk, enable early detection of disease, and improve diagnostic classification. While gene editing methods vary widely, a common characteristic is the need for an automated system for delivering the gene-editing machinery into cells and validation prior to analysis.

In particular, what is needed is a way to process small volumes of material, including genetic material, and/or cells that can be transfected with the genetic material, in an automated or semi-automated manner with a high degree of efficiency and control.

Described herein are methods and apparatuses that may address these needs.

### SUMMARY OF THE DISCLOSURE

The present invention relates to digital microfluidic apparatuses that are capable of high-efficiency poration (electroporation) without compromising the ability to precisely move and process small volumes of material by digital microfluidics. The methods and apparatuses described herein may be used with a digital microfluidics apparatus (system, device, etc.) including in particular those described in any of: U.S. patent application Ser. No. 16/523,876, titled “AIR-MATRIX DIGITAL MICROFLUIDICS APPARATUSES AND METHODS FOR LIMITING EVAPORATION AND SURFACE FOULING,” filed on Jul. 26, 2019; U.S. patent application Ser. No. 16/726,740, titled “FEEDBACK SYSTEM FOR PARALLEL DROPLET CONTROL IN A DIGITAL MICROFLUIDIC DEVICE,” filed on Dec. 24, 2019; U.S. patent application Ser. No. 16/455,459, titled “DIGITAL MICROFLUIDIC DEVICES AND METHODS,” filed on Jun. 27, 2019; U.S. patent application Ser. No. 16/499,681, titled “DIGITAL MICROFLUIDICS APPARATUSES AND METHODS FOR MANIPULATING AND PROCESSING ENCAPSULATED DROPLETS,” filed on Apr. 4, 2018; U.S. patent application Ser. No. 16/614,396, titled “DIGITAL MICROFLUIDICS SYSTEMS AND METHODS WITH INTEGRATED PLASMA COLLECTION DEVICE,” filed on Jul. 23, 2018; and U.S. patent application Ser. No. 16/259,984, titled “DIGITAL MICROFLUIDICS DEVICES AND METHODS OF USING THEM,” filed on Jan. 28, 2019. Each of these applications is herein incorporated by reference in its entirety.

In general, the apparatuses and methods described herein include digital microfluidics systems in which the gap region within which droplets may be driven by electrowetting (e.g., digital microfluidics) by the action of one or more driving electrodes. The driving electrodes are typically separated from the gap region by a dielectric material but may include a dedicated electroporation electrode partially overlaps with the drive electrode. A DMF driving and controlling device may control both movement of one or more droplets within the gap region as well as one or more (preferably an array) of electroporation electrodes within the gap region that are laterally spaced apart from the driving electrodes in an operational configuration.

In some examples, the gap region is formed within a cartridge that may be inserted into the DMF driving and controlling device. In some examples the cartridge does not include the driving electrodes but does include the one or more electroporation electrodes. For example, the bottom of the cartridge on an outside face may be formed of a dielectric material. The one or more electroporation electrodes may be within the gap region of the cartridge on an inner surface of the dielectric material (e.g., one or more sheets of dielectric material). The opposite surface of the dielectric material of the cartridge may be placed against an array of reusable driving electrodes in the DMF driving and controlling device (also referred to herein for convenience as a DMF reader apparatus or a DMF driving and controlling apparatus). Alternatively or additionally, in some examples the array of drive electrodes may be integrated into the cartridge and may include a driving electrode electrical interface on the outside of the cartridge for electrically coupling to the DMF driving and controlling device to drive movement of droplets within the gap region of the cartridge.

In any of the examples described herein the gap may be an air gap. In some examples the gap is an oil-filled gap.

In any of these apparatuses and methods described herein the one or more (e.g., array) of electroporation electrodes within the gap region may be formed on a bottom surface, such as on top of the dielectric material, and within the gap region. The electroporation electrodes may be concurrent with the drive electrodes within the gap, separated by the dielectric material (e.g., sheet), but may not fully overlap. The electroporation electrode(s) may be flat and may extend over at least a portion of the dielectric sheet while leaving one or more gaps so that electrical field generated by the drive electrodes may still drive electrowetting movement of droplets within the gap region. The apparatuses and method described herein illustrate configurations that allow both highly efficient electroporation of cells within a droplet in the gap region, as well as highly effective electrowetting of droplets on/off of these same sites that are configured to provide electroporation.

Also described herein are control configurations in which the electrowetting electrodes may be driven, as controlled by the same DMF driving and controlling device that drives and controls electrowetting, such that the resulting electroporation is both effective and efficient, without damaging the cells being acted upon in the gap region.

Gene editing has emerged to make precise, targeted modifications to genome sequences, and may be relevant to treat a wide range of diseases and disorders. The methods and apparatuses described herein provide a miniaturized digital microfluidic-based platform (which may be referred to as a Miro Flux system) that may be used for gene editing and allows both control of the position(s) of one or more droplets within a gap region but may also concurrently control electroporation within the gap region. These methods and apparatuses provide fast and automated control that may be used for parallel scale processes that may expand the scope of genome editing in a centralized setting.

For example, a digital microfluidics (DMF) cartridge device may include: an upper plate comprising one or more return electrodes; a lower surface comprising a flexible sheet of dielectric material, wherein the flexible sheet of dielectric material forms an outer surface of the cartridge; an air gap formed between the upper plate and the lower surface; and an electroporation electrode attached to the dielectric membrane within the gap region.

In general, the electroporation electrodes are configured so that an underlying drive electrode may apply an electric field through the dielectric to a droplet to move the droplet, even where the electroporation electrode is partially co-extensive with (e.g., atop) the drive electrode under the dielectric sheet. Any of these devices (e.g., cartridges), the electroporation electrode may include a plurality of prongs having exposed regions of dielectric between the prongs. In some examples the electroporation electrode comprises a grid having a plurality of openings therethrough, exposing the underlying flexible sheet of dielectric material. In some examples the electroporation electrode is smaller than the drive electrode. In some examples the electroporation electrode includes one or more openings exposing the underlying dielectric material.

In some examples, the electroporation electrode is adhesively secured to the flexible sheet of dielectric material.

In any of these examples, the electroporation electrode(s) may be positioned at a peripheral region of the air gap. The electrical connector for each electroporation electrode may be connected to a wall of the air gap and to an external connector. Thus, any of these devices may include an electroporation electrode connector on an outer peripheral side of the device.

Any of these electroporation electrodes may have a ramped edge or edges.

The cartridges described herein may include a plurality of spacers within the air gap configured to maintain the spacing between the upper plate and the lower surface.

In general, the cartridges described herein may include a plurality of electroporation electrodes including the electroporation electrode. As mentioned, in some examples the plurality of electroporation electrodes may be arranged around the periphery of the air gap.

In any of these cartridges, the electroporation electrode may have a thickness perpendicular to the flexible dielectric material of less than about 50% of the height of the air gap between the flexible dielectric material and the upper plate.

A digital microfluidics (DMF) cartridge device may include integrated drive electrodes. For example, a device (e.g., cartridge) may include: an upper plate comprising one or more return electrodes; a lower surface comprising a flexible sheet of dielectric material, wherein the flexible sheet of dielectric material comprises an outer surface and an inner surface; an air gap formed between the upper plate and the lower surface; and an electroporation electrode attached to the inner surface of the dielectric membrane within the gap region; and a plurality of drive electrodes on the outer surface of the dielectric membrane, wherein the electroporation electrode extends over one or more of the drive electrodes so that the electroporation electrode covers 80% or less of any of the drive electrodes with the flexible sheet of dielectric therebetween.

Also described herein are systems including any of these cartridges (or for use with any of these cartridges) that may be used for micro-electroporation coordinated with DMF. For example, a digital microfluidics (DMF) system may include: a DMF cartridge comprising: an upper plate comprising one or more return electrodes, a lower surface comprising a flexible sheet of dielectric material forming an outer surface of the cartridge, an air gap formed between the upper plate and the lower surface, and an electroporation electrode attached to the dielectric membrane within the gap region; and a DMF driving and controlling device, the device comprising: a seat configured to receive the outer surface of the cartridge, a plurality of drive electrodes arranged on the seat and configured to seal against the flexible sheet of dielectric material, and an electroporation connector configured to electrically connect to the electroporation electrode when the cartridge is in the seat; wherein the electroporation electrode extends over one or more of the drive electrodes when the cartridge is in the seat so that the electroporation electrode covers 80% or less of any of the drive electrodes with the flexible sheet of dielectric therebetween, so that the plurality of drive electrodes may drive droplets over the electroporation electrodes within the air gap.

Any of these DMF driving and controlling device may include comprises a controller configured to coordinate movement of one or more droplets and electroporation of the one or more droplets. The controller may be configured to apply energy between the electroporation electrode and the return electrode having a pulse time between about 1 to 100 ms, and a voltage between about 0.05 kV to 0.5 kV.

The spacing of the electroporation electrode and the return electrode may be, e.g., between about 250 um-5 mm.

The DMF driving and controlling device may be configured to apply energy between one or more drive electrodes of the plurality of drive electrodes and the one or more return electrodes and to separately apply energy between the electroporation electrodes and the one or more return electrodes. Thus, the same return electrodes may be used for both electroporation and driving DMF. In some examples, the apparatuses described herein may control the electroporation electrodes so that they “float” (e.g., they do not disrupt the electrical energy applied between the drive electrodes and the return electrode(s)).

The DMF apparatus may be configured to apply negative pressure in the seat to secure the flexible sheet to the plurality of drive electrodes.

In some examples the electroporation electrode covers 75% or less (e.g., 70% or less, 65% or less, 60% or less, 55% or less, 50% or less, 45% or less, 40% or less, etc.) of any of the drive electrodes.

The systems described herein may use any of the devices (e.g., cartridges) described herein.

Also described herein are methods of using any of these apparatuses (e.g., systems and devices) for micro-electroporation. For example, a micro-electroporation method may include: applying energy to one or more driving electrodes to drive a droplet of liquid within an air gap of a DMF cartridge so that the droplet is over an electroporation region; electroporating the droplet by applying energy to an electroporation electrode within the air gap, wherein the electroporation electrode partially overlies a drive electrode in the electroporation region, further wherein 80% or less of the drive electrode in the electroporation region is covered by the electroporation electrode with a sheet of dielectric material between the electroporation electrode and the drive electrode in the electroporation region; and applying energy to the drive electrode in the electroporation region to move the droplet off of the electroporation electrode.

All of the methods and apparatuses described herein, in any combination, are herein contemplated and can be used to achieve the benefits as described herein.

All of the methods and apparatuses described herein, in any combination, are herein contemplated and can be used to achieve the benefits as described herein.

### BRIEF DESCRIPTION OF THE DRAWINGS

A Better Understanding of the Features and Advantages of the Methods and Apparatuses Described Herein Will be Obtained by Reference to the Following Detailed Description that Sets Forth Illustrative Embodiments, and the Accompanying Drawings of which:

FIG. 1A shows one example of a DMF driving and controlling apparatus and a cartridge configured to provide both DMF and micro-electroporation as described herein.

FIG. 1B shows an example of a DMF cartridge being inserted into a DMF driving and controlling apparatus to drive both DMF and micro-electroporation.

FIG. 2A schematically illustrates a section through one example of a cartridge configured for both DMF and micro-electroporation as described herein. In FIG. 2A the cartridge is shown seated in a portion of a DMF driving and controlling apparatus.

FIGS. 2B-2D show side, top and back views, respectively of an example of a cartridge and DMF driving and controlling apparatus configured for both DMF movement of droplets and micro-electroporation.

FIGS. 3A-3B illustrate comparisons of the efficacy of bottom-to-top (FIG. 3A) and side-to-side (FIG. 3B) micro-electroporation configurations as described herein.

FIG. 4A is a table showing that typical DMF parameters do not significantly reduce viability of cells to be transfected.

FIG. 4B is a table showing that these same typical DMF parameters used in FIG. 4A are not sufficient to result in electroporation.

FIG. 5A shows a first example of electroporation electrodes having prongs as described herein.

FIG. 5B shows a second example of electroporation electrodes having prongs as described herein.

FIG. 6A shows an example of electroporation electrodes having a mesh structure as described herein.

FIG. 6B shows an enlarged view of an electroporation electrode of FIG. 6A.

FIG. 7 is a table illustrating one example of the methods described herein (using a square wave with various parameters as described herein) to transfect by micro-electroporation.

FIG. 8 is a chart illustrating the percent of transfection and/or percent of viability of cells by pulse time for square wave pulses applied, similar to the data shown in FIG. 7.

FIGS. 9A-9D are graphs showing percentage of viability and percentage of transfection by pulse time for square pulses at different voltage densities (e.g., 1 kV/cm in FIG. 9A, 2 kV/cm in FIG. 9B, 3 kV/cm in FIG. 9C and 4 kV/cm in FIG. 9D).

FIG. 10 is a table illustrating one example of the methods described herein (using exponential decay (exp) pulses with various parameters as described herein) to transfect by micro-electroporation.

FIG. 11 is a chart illustrating the percent of transfection and/or percent of viability of cells by pulse time for exp pulses applied, similar to the data shown in FIG. 10.

FIGS. 12A-12D are graphs showing percentage of viability and percentage of transfection by pulse time for exp pulses at different voltage densities (e.g., 1 kV/cm in FIG. 12A, 1.5 kV/cm in FIG. 12B, 2 kV/cm in FIG. 12C and 3 kV/cm in FIG. 12D).

### DETAILED DESCRIPTION

Described herein are methods and apparatuses for micro-electroporation in conjunction with digital microfluidic (DMF) to control one or more droplets. These methods and apparatuses may include both devices and systems, such as cartridges, DMF driving and controlling devices, etc.). Although the methods and apparatuses described herein may be specifically adapted for air gap DMF apparatuses (also referred to herein as air matrix DMF apparatuses), these methods and apparatus may be configured for use in other DMF apparatuses (e.g., oil-filled gap, etc.).

In general, these apparatuses provide an arrangement of electroporation electrodes within the gap (e.g., air gap) region that partially overlap one or more electrowetting electrodes, but that are separated from the one or more electrowetting electrodes (“drive electrodes”) by a dielectric material, such as a sheet or sheets of dielectric material. In some examples the gap region is formed as part of a cartridge that does not include the drive electrodes, but instead are part of the DMF driving and controlling device that receives the cartridge; alternatively, the cartridge including the gap region may include an array of drive electrodes that may be controlled by the DMF driving and controlling device.

For example, a cartridge for a digital microfluidics (DMF) apparatus may have a bottom and a top and may include: a sheet of dielectric material having a first side and a second side, the first side forming an exposed bottom surface on the bottom of the cartridge, wherein at least the second side of the sheet of dielectric material comprises a first hydrophobic surface. The cartridge may also include a top plate having a first side and a second side. All or a portion of the top plate (which may form the upper surface of the gap region in the cartridge) may be configured as a ground electrode of the top plate. For example, the ground electrode may comprise a grid pattern forming a plurality of open cells. The upper surface (including any ground/return electrodes) in the gap region of the cartridge may include a hydrophobic layer. The hydrophobic layer may cover the ground electrode. The cartridge also typically includes the gap region separating a bottom surface, which may also include a hydrophobic layer from the top, or roof layer (which may also include a hydrophobic layer). Within the gap region one or more electrowetting electrodes may be included and configured so that they do not fully overlap with the underlying drive electrode(s) when the device is to be operated by the DMF driving and controlling apparatus.

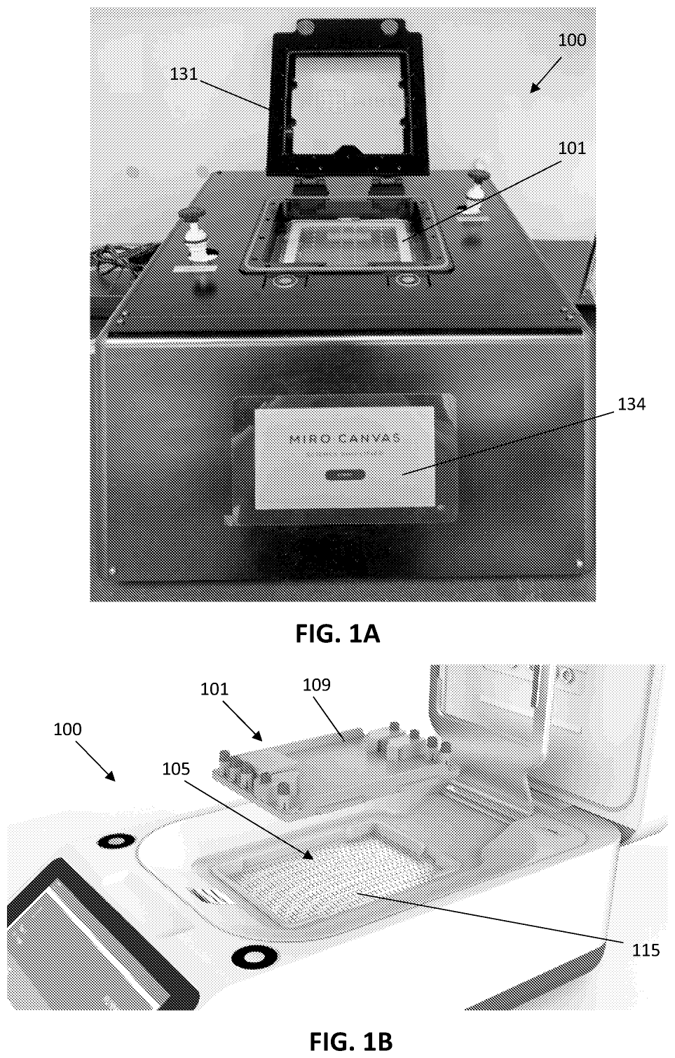

FIG. 1A shows one example of a DMF driving and controlling apparatus 100. A cartridge 101 is shown within the apparatus. In this example, the cartridge may include both an array of drive electrodes arranged over the bottom of the cartridge and one or more micro-electroporation electrodes that are arranged opposite one or more drive electrodes, separated by a dielectric. As will be described in greater detail below, the electroporation electrodes may have a smaller surface area that the drive electrodes and may only partially overlap (e.g., cover) the drive electrodes. The electroporation electrode(s) may be within the gap (e.g., air gap) of the cartridge. The electroporation electrode(s) may be configured specifically for efficient electroporation without harming or damaging (e.g., killing) the cells within the droplet. Alternatively, the DMF driving and controlling apparatus 100 in FIG. 1A may be configured to receive a cartridge that does not include the array of drive electrodes; instead, the array of drive electrodes may be positioned within the DMF driving and controlling apparatus and may be reusable. The DMF driving and controlling apparatus in FIG. 1A may also include a vacuum or suction (e.g., negative pressure) system for retaining the cartridge within the seating region of the apparatus (not shown) and may include a controller having control circuitry for coordinating both the electrowetting (DMF) and the electroporation. The DMF driving and controlling apparatus may include an electrical interface region for interfacing with the one or more (e.g., array) of micro-electrowetting electrodes within the gap region of the cartridge. The device also includes a lid 131 that is hinged to the body to secure the cartridge within the seating region in the desired configuration and/or to cover the cartridge. The apparatus also includes a user interface 134. The controller may include one or more processors and may include communication (e.g., wireless communication) circuitry for sending and/or receiving information between the apparatus and remote processor or server.

FIG. 1B shows another example of an apparatus that may be used with a cartridge 101 as described herein. The cartridge in this example does not include the drive electrodes but does include the one or more electrowetting electrodes within the gap region. The upper (return) electrodes in any of these cartridges may be used as the return electrode(s) for both the electrowetting and for the electroporation. In FIG. 1B, the cartridge is shown being inserted into the DMF driving and controlling apparatus 100 that includes a seating region or base 105 including an array of drive electrodes 115. The cartridge may include a cartridge body 109 having one or more microfluidic channels and/or chambers for dispensing or receiving fluid into/out of the air gap, which is bounded on the side facing the cartridge body.

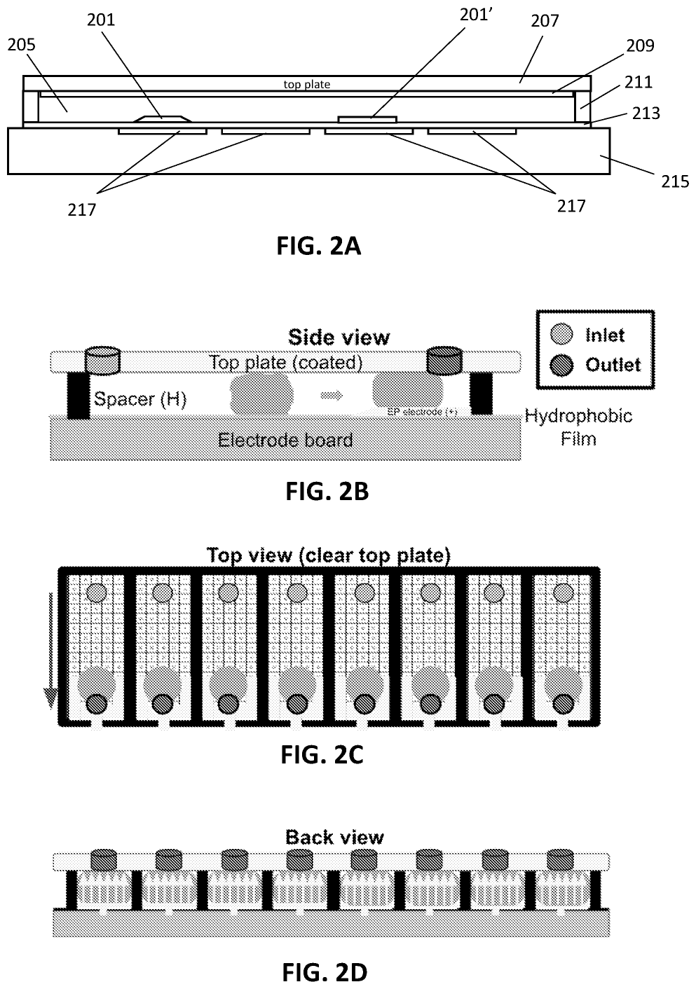

FIG. 2A shows a section through one example of a section through a cartridge including a plurality of micro-electroporation electrodes 201, 201′ within the gap region 205. The gap region in this example is shown as an air gap (e.g., the cartridge is for an air matrix device). In FIG. 2A, the cartridge also includes a top plate 207 holding a return or ground electrode 209. In this example a single return/ground electrode is shown, which may be formed by a coating, such as a conductive coating (e.g., ITO). The top plate 207 is spaced apart from the bottom of the cartridge to form the air gap region 205 by spacers 211 that may form the height (H) of the air gap. In some cases, the spacers and/or top plate may be formed as a frame. The bottom of the cartridge in this example is a sheet of dielectric material 213; the micro-electroporation electrodes (also referred to herein as electroporation electrodes or EP electrodes) 201, 201′ are attached. Both the dielectric and/or the micro-electroporation electrodes may be coated with a hydrophobic coating. Similarly, the top plate and/or return electrode 209 may be coated with a hydrophobic coating. The top plate may include one or more openings (inlets and/or outlets, not shown) including for adding and/or removing fluid droplets into the gap region 205.

In FIG. 2A, the cartridge sits on top of a seating surface of a DMF driving and controlling apparatus 215 that includes an array of driving electrode 217 arranged within the seating surface. In some examples the cartridge may include the drive electrodes integrated into the cartridge, as described above.

Although the section through the example in FIG. 2A shows only two electroporation electrodes, any number of electroporation electrodes may be included (e.g., 2, 3, 4, 5, 6, 7, 8, 9, 10, 12, 15, 20, 24, 28, 30, 32, 36, 38, 40, 50, 60, 64, 70, 75, 80, 84, 90, 96, etc. such as 10 or more, 15 or more 20 or more, 25 or more, 30 or more, 40 or more, 50 or more, 60 or more, 70 or more, 80 or more, 90 or more 96 or more, 100 or more, etc.). Each of the electroporation electrodes may include an electrical connection (not shown in FIG. 2A). The electrical connections to individual electroporation electrodes may be separate or may be combined (e.g., so that multiple electroporation electrodes may be concurrently actuated). The electrical connections may connect to an electrical interface on an outer surface of the cartridge that may couple to the DMF driving and controlling apparatus. In some examples, the one or more electroporation electrodes may be arranged near the periphery of the cartridge (e.g., of the gap region of the cartridge) so that the electoral connections (leads) connecting the electroporation electrodes to the connector on the outer region of the cartridge may not interfere with droplet movement within the gap region. Alternatively in some examples the electrical connection(s) to the electroporation electrodes (including more centrally positioned electroporation electrodes) may be coupled through a spacer extending across the gap region (not shown).

FIG. 2A is shown as a schematic; dimensions of the components may be different. In FIG. 2A two different examples of electroporation electrodes are shown. In one example, the edges of the electroporation electrode 201 may be curved or beveled, which may prevent showing a steep edge to the aqueous droplet. Alternatively in some examples the electroporation electrode has a squared-off edge 201′. The electroporation electrodes may have an unbroken, solid appearance (e.g., a square, or other shape) or they may include openings (e.g., a grid or other pattern) exposing the dielectric region beneath the electroporation electrode. Examples of this are provided below. In the example shown in FIG. 2A, the surface area of the electroporation electrode covering the dielectric sheet forming the bottom of the gap region is less than the surface area exposed to the underlying drive electrode 217 beneath the dielectric when the cartridge is seated in the DMF driving a controlling apparatus (or when the drive electrodes are integrated into the cartridge). In general, the electroporation electrode may cover 95% or less of the underlying drive electrode (e.g., 90% or less, 85% or less, 80% or less, 75% or less, 70% or less, 65% or less, 60% or less, 55% or less, 50% or less, 45% or less, 40% or less, 35% or less, etc.). This may allow both driving and electroporation to be performed in the same location of the gap region without interference between the two electrodes, and without requiring different driving energy (voltage, etc.) to be applied to those drive electrodes overlapping with electroporation electrodes.

In general, the DMF-based apparatuses described herein are configured for miniaturizing electroporation (EP) reactions, including CRISPR editing. This may reduce the need for expensive reagents and may minimize the number of cells per reaction. The apparatuses described herein may utilizes electromechanical forces for manipulating (i.e., moving, merging, mixing, dispensing, etc.) microliter to nanoliter volumes of fluids across the bottom surface of the cartridge in an automated fashion.

FIGS. 2B-2D show another example of a portion of a cartridge similar to that shown in FIG. 2A. FIG. 2B shows a side view, FIG. 2C shows a top view, and FIG. 2D shows a back view. The example cartridge shown in FIG. 2C was configured to perform early stage feasibility tests.

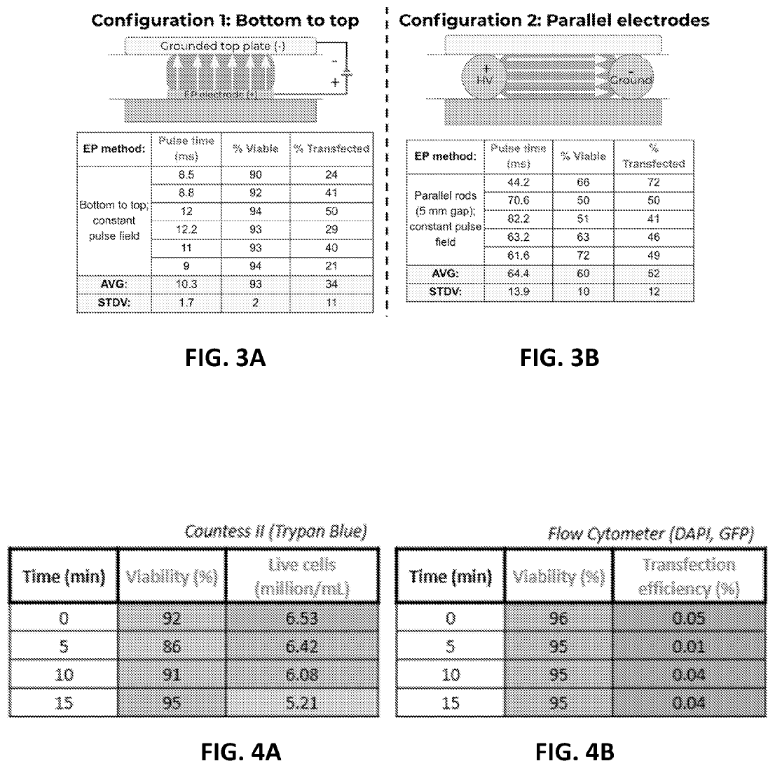

In one example, the results of which are illustrated in FIGS. 3A and 3B, K562 cells were modified using a GFP buffer kit (Lonza). Editing results were measured using the iQue3 flow cytometer (Sartorius) and analyzed in-house (FlowJo). Transfection of K562 cells was successfully achieved on the prototype instrument. Two DMF electroporation (EP) configurations were tested for feasibility and consistency using Fluorescence-Activated Cell Sorting cytometer. The same cell input, buffer and pulse input parameters were used to test the reproducibility of each configuration (pulse input parameters were not optimized for this test). In this example, the two different configurations examined were the relative orientation of the electroporation electrodes, arranged as either bottom-to-top (FIG. 3A and as shown in FIGS. 2A-2B) or a side-to-side (also referred to herein as a parallel wires configuration, as shown in FIG. 3B). The bottom-to-top configuration performed better in that this configuration consistency had a lower standard deviation across experiments. For example, as shown in FIG. 3A, a relatively lower electroporation pulse time (e.g., 8.5 ms, 12 ms, 12.2 ms, 11 ms, 9 ms), resulted in greater than 90% viability in every case with an average percent transfection between 21-50% (average of 34%). In contrast, although the percent transfection of the side-by-side configuration was somewhat higher (average of 52%), the percent viability was substantially lower (average of 60%).

The DMF micro-electroporation apparatuses and methods described herein may provide unique pulses to achieve transfection as compared to traditional electroporation. The parameters tested were the pulse time, adjusted from 1 to 100 ms, the input voltage, adjusted from 0.05 kV to 0.5 kV, and the distance between electrodes, adjusted from 250 um-5 mm. After iterating through a range of parameters, more than about 85% transfection was achieved with more than about 90% viability in both configurations.

Because the top-to-bottom configuration may interfere less with the gap region and the space for DMF manipulation, the top-to-bottom arrangement may be used in many cases. However, the configuration and position of the electroporation electrode may be controlled to prevent the electroporation electrode(s) from interfering with electrowetting by one or more underlying electrode.

Note that although FIG. 2A shows electroporation electrodes that are smaller than the underlying DMF driving electrodes, in some examples the underlying DMF electrodes may instead be configured to overlay more than one drive electrode, as shown in FIG. 2B. in this example, the electroporation electrode(s) may instead include one or more openings exposing the dielectric material and allowing the underlying drive electrode(s) to drive droplet movement by electrowetting. For example, the electroporation electrode may include one or more windows arranged to be above each of the underlying drive electrodes (e.g., exposing the dielectric material above each).

In any of these examples the method and apparatus may adjust the electroporation electrode when driving droplet movement via driving electrodes so that the intervening electroporation does not act as return electrode (e.g., it may electrically float or may be set to a predetermined, including in some examples, neutral, value). The electroporation electrode is generally configured so as to be completely co-extensive with the underlying drive electrode. In general, an electroporation electrode may be configured and/or arranged so that less than 20% (e.g., less than 25%, less than 30%, less than 35%, less than 40%, less than 45%, less than 50%, less than 55%, less than 60%, less than 65%, less than 70%, less than 75%, less than 80%, less than 85%, less than 90%, etc.) of each underlying drive electrode is occluded when the device is seated in the DMF driving and controlling apparatus.

In some examples the DMF driving and controlling apparatus may apply different energy to the drive electrodes that are partially occluded by one or more electroporation electrodes. For example, the DMF driving and controlling apparatus may apply a greater drive energy to the partially occluded drive electrodes.

The electroporation electrode(s) may be attached to the dielectric layer by an adhesive. The adhesive (or any other configuration) may prevent fluid from becoming trapped beneath the electroporation electrode. In some examples the electroporation layer is not adhesively connected, but rests on the dielectric layer. The electroporation electrode may be applied to the dielectric layer when the dielectric layer has been pretensioned.

As mentioned above, any appropriate number of electroporation electrodes may be included in each cartridge and coordinated by the DMF driving and controlling apparatus. For example, a cartridge may include 64 electroporation sites (e.g., electrodes); in some examples the cartridge includes 96 electroporation sites (e.g., electrodes).

The electroporation electrode may extend into the gap region by less than a maximum percentage. For example, the thickness of the electroporation electrode may be selected so that it intrudes 60% or less (e.g., 55% or less, 50% or less, 45% or less, 40% or less, 35% or less, 30% or less, 25% or less, 20% or less, 15% or less, etc.). For example, the electroporation electrode may be between about 500 nm to 2.5 mm (e.g., with a 5 mm gap spacing).

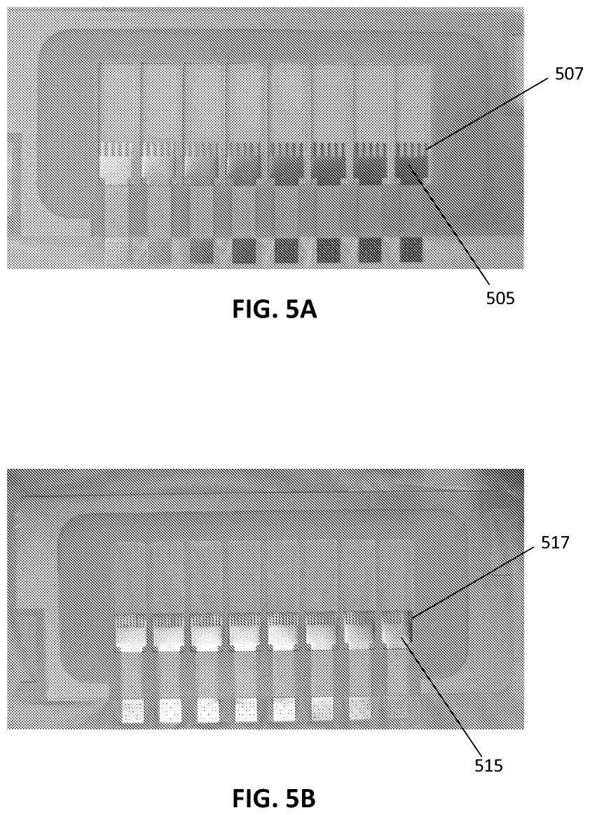

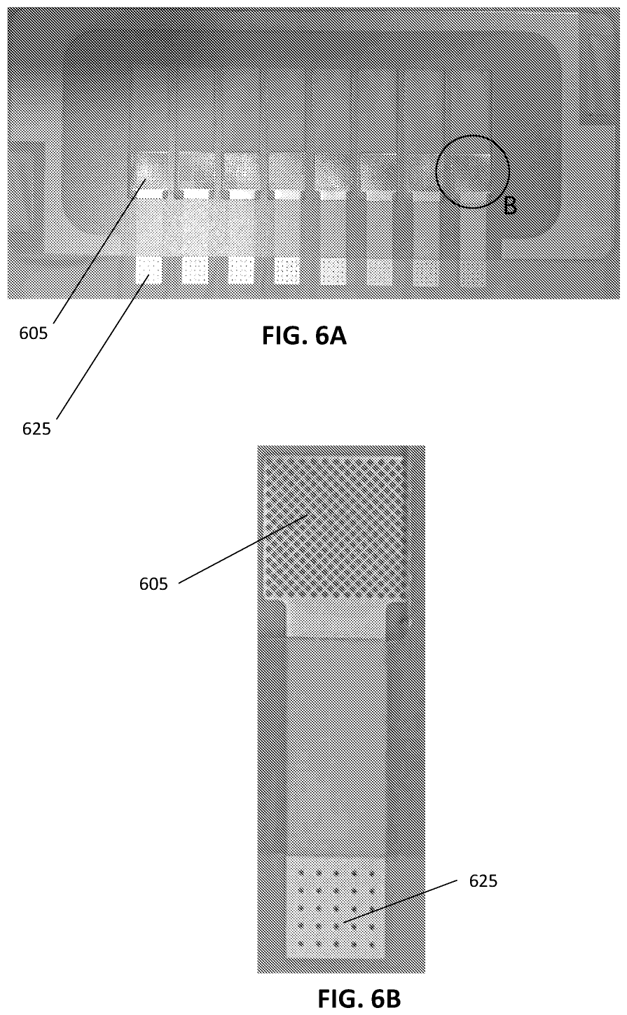

FIGS. 5A-5B and 6A-6B illustrate examples of electroporation electrodes that may be used. For example, FIG. 5A shows one example of an array of electroporation electrodes 505 that each include a plurality of prongs or arms 507 (having fork-like appearance). The resulting prongs (e.g., trident shaped electrodes) include regions exposing the underlying dielectric layer. FIG. 5B shows example electroporation electrodes 515 including ten prongs 517, whereas FIG. 5A shows an example having 5 prongs. Any appropriate number of prongs (and therefore gaps) may be used.

FIGS. 6A and 6B illustrate an example in which the electroporation electrode 605 is configured as a mesh. In this example, the mesh includes a plurality of openings. The mesh may have any appropriate opening size (pore size), so as to expose the desired amount of dielectric underneath the electroporation electrode, as described above. In FIG. 6B the example electrode is a 25% mesh wire, covering only 25% of the underlying dielectric. The electrodes may be formed of any appropriate material. In the example shown the electrodes are formed of Cu. In FIG. 6A-6B, the electroporation electrodes are between 0.025 mm thick or less. Each electrode 605 also include a connector with a contact 625 that may be positioned on an outer surface of the cartridge, as described above.

In general, the DMF driving apparatus alone does not provide sufficient energy (using the drive electrodes) through the dielectric material to cause electroporation. This was tested using a prototype DMF driving and controlling apparatus and cartridge. For example, FIG. 4A illustrates the effect of DMF generally on the viability of a sample of cells (40 μL droplet) to which DMF alone was applied for various times (between 0-15 min). Essentially no effect was seen on cell viability. Similarly, when DMF was applied with a CFP material within the liquid droplet, the application of DMF alone did not result in transfection (e.g., electroporation) of the cells within the droplet, as illustrated in FIG. 4B. Thus, the energy applied for DMF were not sufficient to transfect cells.

In general, the DMF driving and controlling apparatuses described herein are configured to integrate driving of droplets within the cartridge with electroporation. For example, any of the DMF driving and controlling apparatuses described herein may include a dual driving system including control circuitry to control voltage output, pulse and frequency of the electroporation electrode(s), which may be user-defined and/or adjusted, as well as control circuitry to apply energy to the drive electrode in sequence to drive droplets.

The DMF driving and controlling apparatus may automate all of the step required for electroporation, allowing manipulation of droplets as low as 1.2 uL, and may be scalable to allow for high-throughput reactions (greater than 48 reactions in parallel).

### EXAMPLES

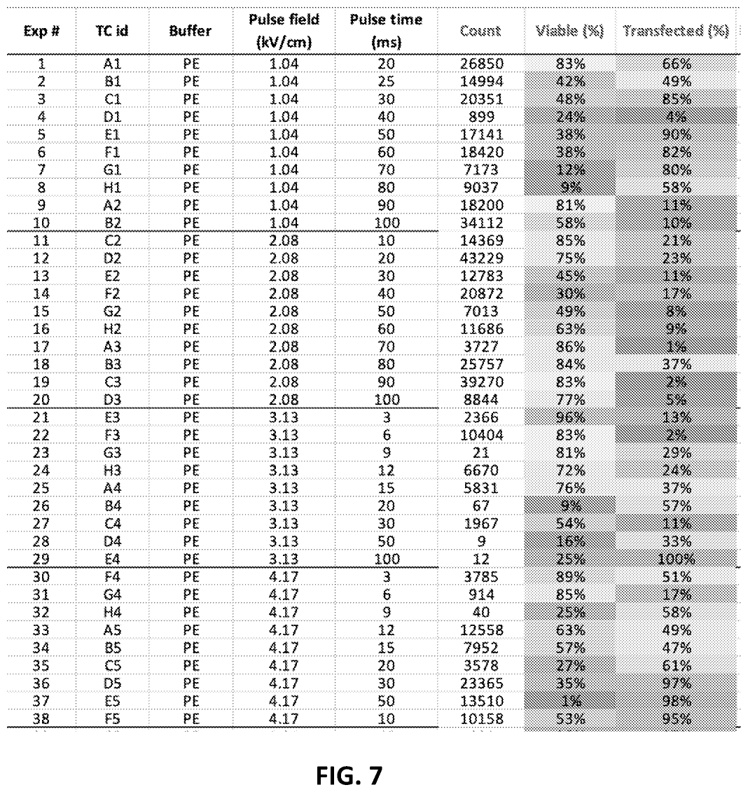

FIGS. 7, 8 and 9A-9D illustrate one example of an electroporation method in which energy is applied to one or more driving electrodes to drive a droplet of liquid within an air gap of a DMF cartridge so that the droplet is over an electroporation region. The droplet includes cells suspended within a buffer (e.g., PE buffer). The cells are included in the droplet in a density of 40 M/ml for a 10 uL reaction volume (droplet); thus approximately 0.4 M of cells are used per reaction. Droplets include 1 uL of tetradecane. The air gap has a gap size of approximately 0.048 cm.

Each droplet is electroporated within the air gap by applying energy to an electroporation electrode within the air gap. The electroporation electrode partially overlies a drive electrode in the electroporation region (as illustrated above). Approximately 80% or less of the drive electrode in the electroporation region is covered by the electroporation electrode with a sheet of dielectric material between the electroporation electrode and the drive electrode in the electroporation region. Following the application as specified (the amount and waveform of the electroporation energy applied to the electrode within the air gap is varied as indicated in the figures, energy is applied to the drive electrode in the electroporation region to move the droplet off of the electroporation electrode.

For example, FIG. 7 is a table showing the results of 38 experiments with droplets including 0.4 M of cells. Different pulse fields (between about 1 and about 4 kV/cm were examined) and different pulse times (between about 3 ms and 100 ms were examined) and cells were thereafter examined to determine viability (percent viability) and transfection (percent transfection). Transfection could be determined based on expression of, e.g., transfected marker (e.g., green florescent protein).

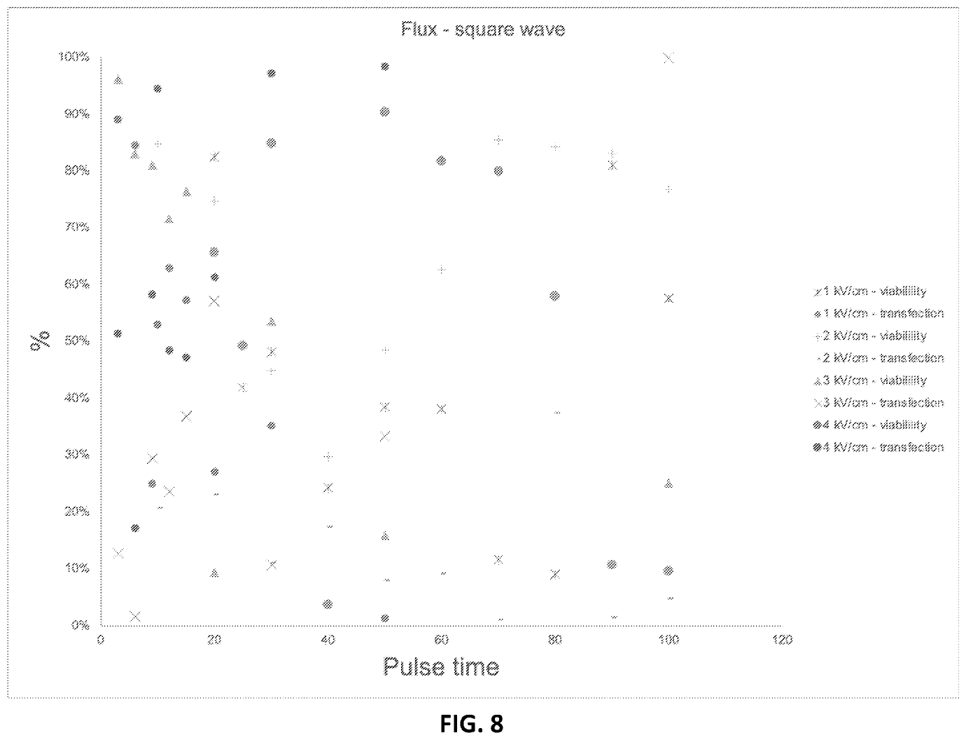

As shown in FIG. 7, transfection using a square pulse of about 1 kV/cm for about 20 ms and/or for about 4 kV/cm and between 3-15 ms resulted in a relatively % viability and % transfection. The graph shown in FIG. 8 summarizes these findings. In general, lower pulse times and higher pulse fields were more successful (both higher percent viability and percent transfection) in these examples.

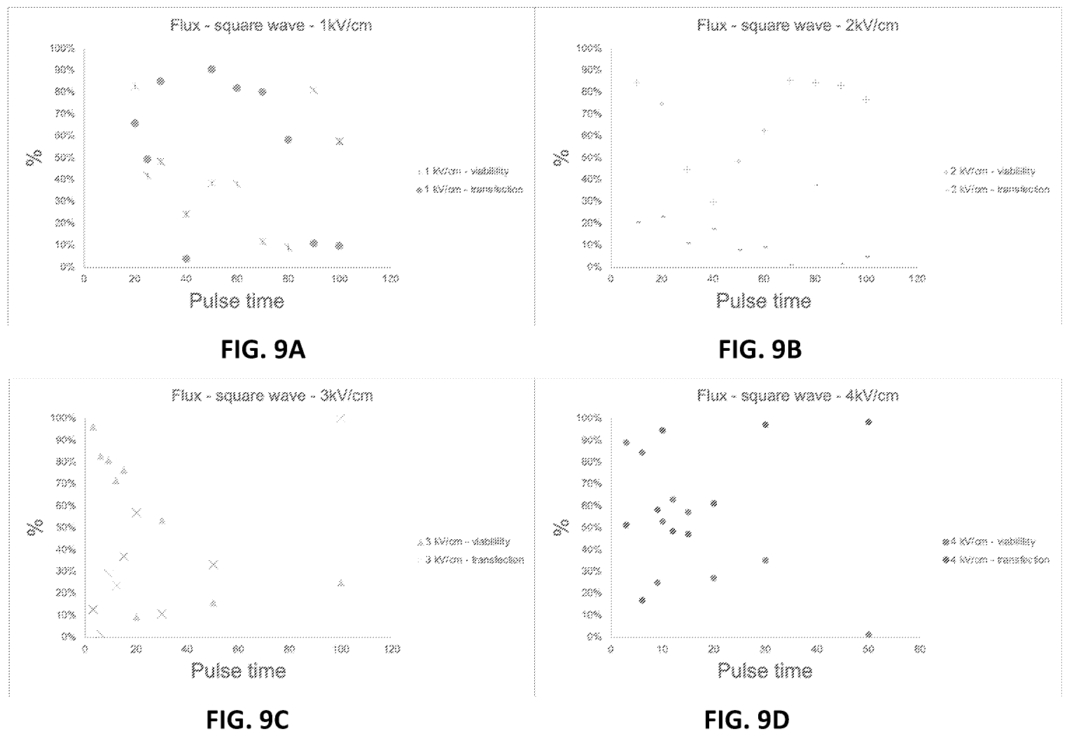

FIGS. 9A-9D illustrate specific pulse fields (e.g., 1 kV/cm, 2 kV/cm, 3 kV/cm and 4 kV/cm) for individual examples.

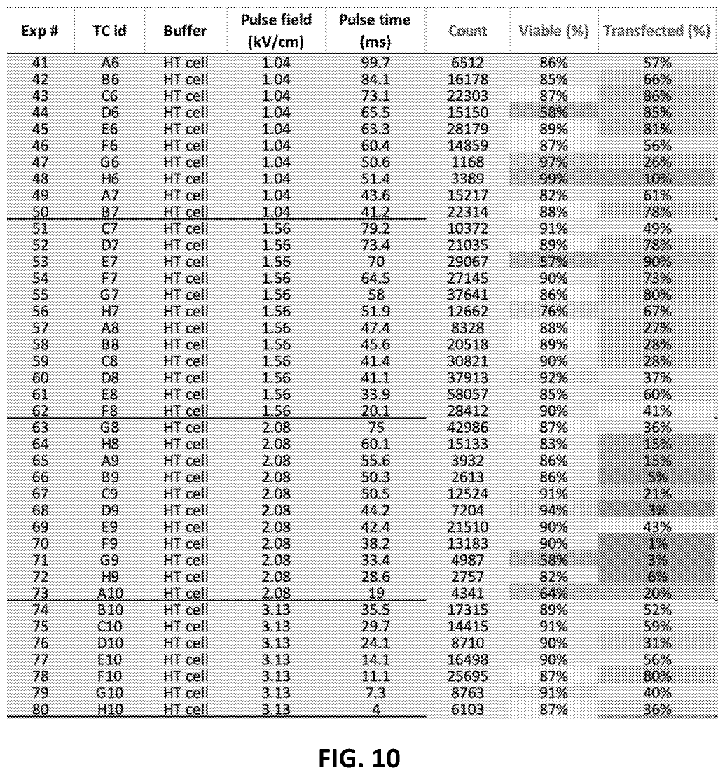

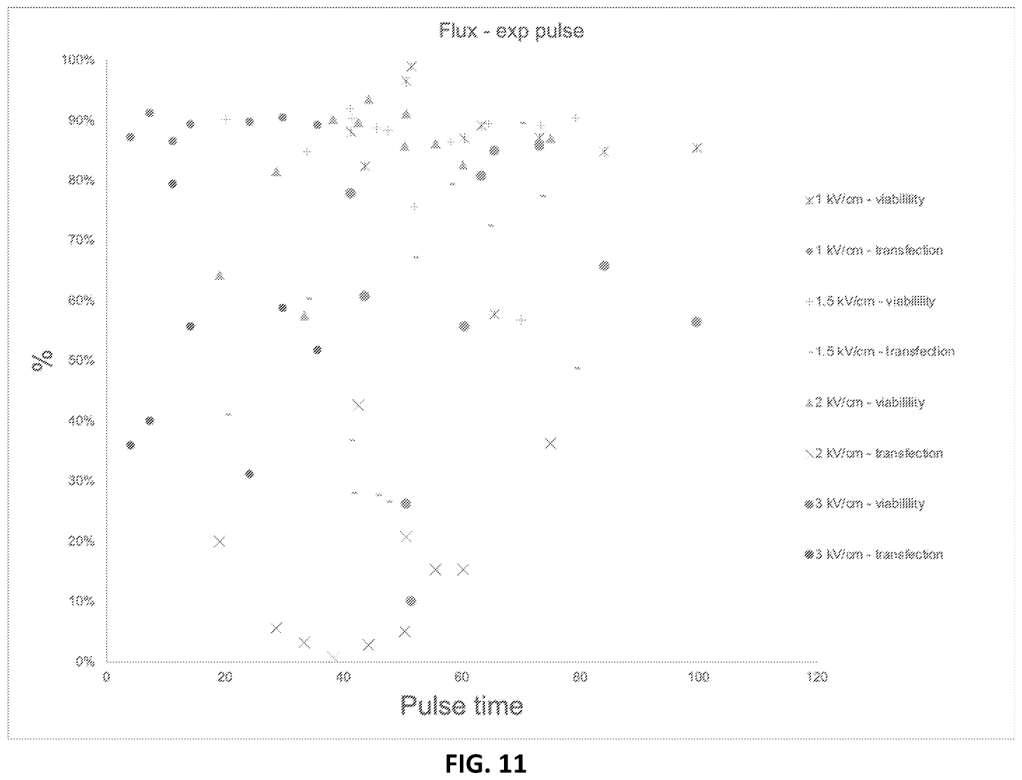

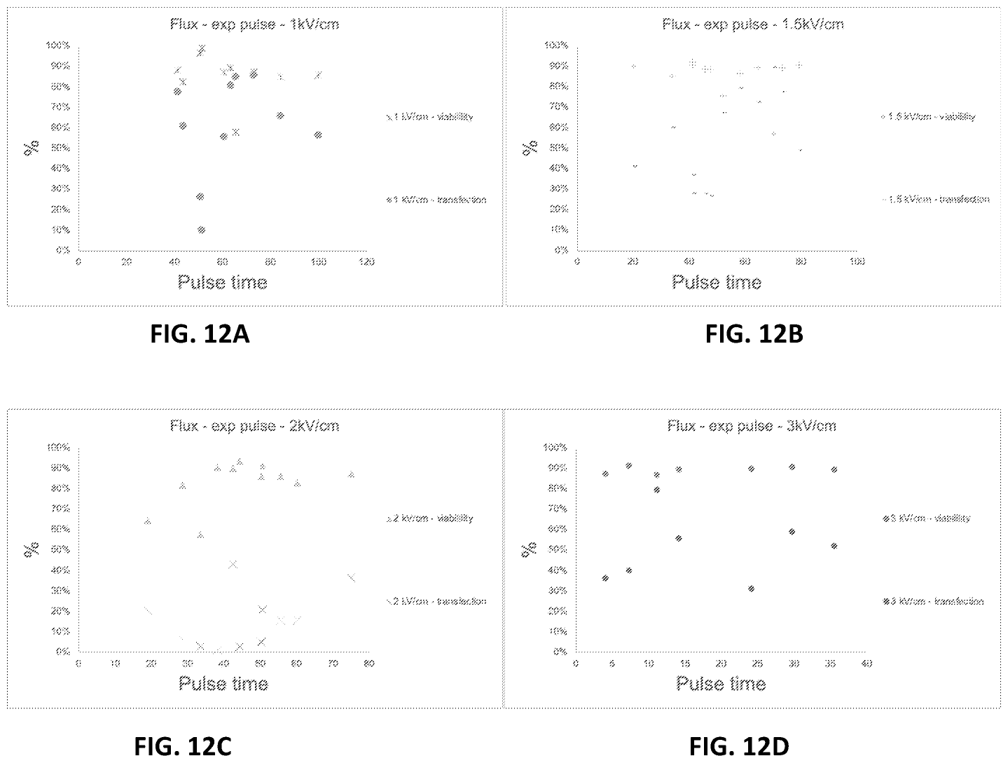

Similar results were found when using an exponentially decaying pulse, as shown in FIGS. 10, 11 and 12A-12D. In FIG. 10, the table shows the results on 39 experiments using exponential decay electroporation waveforms that were applied at a pulse field of about 1 kV/cm, 1.5 kV/cm, 2 kV/cm or 3 kV/cm (e.g., between 1-3 kV/cm) and pulse times between about 4 ms and 100 ms (e.g., 99.7 ms). As shown, in general, the exponential waveforms used had fairly high viability across all conditions, however the percent transfection varied between almost entirely un-transfected (e.g., at 2 kV/cm) and highly transfected (at 1 kV/cm). FIG. 11 graphically summarizes this data, and FIGS. 12A-12D illustrate specific results for both percent transfection and percent survivability following the treatment with an exponentially decaying waveform as described. In both square pulses and exponentially decaying waveforms there were successful transfections using this technique, including those having a very high success rate. Generally speaking the intermediate range of about 2 kV/cm was the least successful, while either the low end (e.g., around about 1 kV/cm, e.g., between 0.1 kV/cm and 1.5 kV/cm or a mid-range (e.g., between about 3 and about 5 kV/cm) also had both relatively high transfection and viability.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A digital microfluidics (DMF) cartridge device, the device comprising:
an upper plate comprising one or more return electrodes;
a lower surface comprising a flexible sheet of dielectric membrane, wherein the flexible sheet of dielectric membrane forms an outer surface of the cartridge;
an air gap formed between the upper plate and the lower surface; and
an electroporation electrode attached to the dielectric membrane within the gap region.

2. The device of claim 1, wherein the electroporation electrode comprises a plurality of prongs having exposed regions of the dielectric membrane between the prongs.

3. The device of claim 1, wherein the electroporation electrode comprises a grid having a plurality of openings therethrough, exposing the underlying flexible sheet of dielectric membrane.

4. The device of claim 1, wherein the electroporation electrode is adhesively secured to the flexible sheet of dielectric membrane.

5. The device of claim 1, wherein the electroporation electrode is positioned at a peripheral region of the air gap.

6. The device of claim 1, wherein the electroporation electrode has ramped edges.

7. The device of claim 1, further comprising an electroporation electrode connector on an outer peripheral side of the device.

8. The device of claim 1, further comprising a plurality of spacers within the air gap configured to maintain the spacing between the upper plate and the lower surface.

9. The device of claim 1, further comprising a plurality of electroporation electrodes including the electroporation electrode.

10. The device of claim 9, wherein the plurality of electroporation electrodes are arranged around the periphery of the air gap.

11. The device of claim 1, wherein the electroporation electrode has a thickness perpendicular to the flexible dielectric membrane of less than about 50% of the height of the air gap between the flexible dielectric membrane and the upper plate.

12. A digital microfluidics (DMF) cartridge device, the device comprising:
an upper plate comprising one or more return electrodes;
a lower surface comprising a flexible sheet of dielectric membrane, wherein the flexible sheet of dielectric membrane comprises an outer surface and an inner surface;
an air gap formed between the upper plate and the lower surface; and
an electroporation electrode attached to the inner surface of the dielectric membrane within the gap region; and
a plurality of drive electrodes on the outer surface of the dielectric membrane,
wherein the electroporation electrode extends over one or more of the drive electrodes so that the electroporation electrode covers 80% or less of any of the drive electrodes with the flexible sheet of dielectric membrane therebetween.

13. A digital microfluidics (DMF) system, the system comprising:
a DMF cartridge comprising: an upper plate comprising one or more return electrodes, a lower surface comprising a flexible sheet of dielectric membrane forming an outer surface of the cartridge, an air gap formed between the upper plate and the lower surface, and an electroporation electrode attached to the dielectric membrane within the gap region; and
a DMF driving and controlling device, the device comprising:
a seat configured to receive the outer surface of the cartridge,
a plurality of drive electrodes arranged on the seat and configured to seal against the flexible sheet of dielectric membrane, and
an electroporation connector configured to electrically connect to the electroporation electrode when the cartridge is in the seat;
wherein the electroporation electrode extends over one or more of the drive electrodes when the cartridge is in the seat so that the electroporation electrode covers 80% or less of any of the drive electrodes with the flexible sheet of dielectric membrane therebetween, so that the plurality of drive electrodes may drive droplets over the electroporation electrodes within the air gap.

14. The system of claim 13, wherein the DMF driving and controlling device comprises a controller configured to coordinate movement of one or more droplets and electroporation of the one or more droplets.

15. The system of claim 14, wherein the controller is configured to apply energy between the electroporation electrode and the return electrode having a pulse time between about 1 to 100 ms, and a voltage between about 0.05 kV to 0.5 kV.

16. The system of claim 13, wherein the spacing of the electroporation electrode and the return electrode is between about 250 um-5 mm.

17. The system of claim 13, wherein the DMF driving and controlling device is configured to apply energy between one or more drive electrodes of the plurality of drive electrodes and the one or more return electrodes and to separately apply energy between the electroporation electrodes and the one or more return electrodes.

18. The system of claim 13, wherein the DMF driving and controlling device is configured to apply negative pressure in the seat to secure the flexible sheet to the plurality of drive electrodes.

19. The system of claim 13, wherein the electroporation electrode covers 50% or less of any of the drive electrodes.

20. The system of claim 13, wherein the electroporation electrode comprises a plurality of prongs having exposed regions of the dielectric membrane between the prongs.

21. The system of claim 13, wherein the electroporation electrode comprises a grid having a plurality of openings therethrough, exposing the underlying flexible sheet of the dielectric membrane.

22. The system of claim 13, wherein the electroporation electrode is adhesively secured to the flexible sheet of the dielectric membrane.

23. The system of claim 13, wherein the electroporation electrode is positioned at a peripheral region of the air gap.

24. The system of claim 13, wherein the electroporation electrode has ramped edges.

25. The system of claim 13, further comprising an electroporation electrode connector on an outer peripheral side of the device.

26. The system of claim 13, further comprising a plurality of spacers within the air gap configured to maintain the spacing between the upper plate and the lower surface.

27. The system of claim 13, further comprising a plurality of electroporation electrodes including the electroporation electrode.

28. The system of claim 27, wherein the plurality of electroporation electrodes is arranged around the periphery of the air gap.

29. The system of claim 13, wherein the electroporation electrode has a thickness perpendicular to the flexible dielectric membrane of less than about 50% of the height of the air gap between the flexible dielectric membrane and the upper plate.

30. A micro-electroporation method, the method comprising:
applying energy to one or more driving electrodes to drive a droplet of liquid within an air gap of a DMF cartridge so that the droplet is over an electroporation region;
electroporating the droplet by applying energy to an electroporation electrode within the air gap, wherein the electroporation electrode partially overlies a drive electrode in the electroporation region, further wherein 80% or less of the drive electrode in the electroporation region is covered by the electroporation electrode with a sheet of dielectric membrane between the electroporation electrode and the drive electrode in the electroporation region; and
applying energy to the drive electrode in the electroporation region to move the droplet off of the electroporation electrode.
